(19) United States
(12) Patent Application Publication
(10) Pub. No.: US 2020/0176085 A1
(43) Pub. Date: Jun. 4, 2020

(54) DETERMINING PHENOTYPE FROM GENOTYPE

(71) Applicant: Julian GOUGH, Bristol (GB)

(72) Inventors: Julian GOUGH, Bristol (GB); Jan ZAUCHA, Bristol (GB); Natalie THURLBY, Bristol (GB)

(73) Assignee: Julian GOUGH, Bristol (GB)

(21) Appl. No.: 16/070,920

(22) PCT Filed: Jan. 18, 2016
(86) PCT No.: PCT/IB2016/000195

§ 371 Date: Jul. 18, 2018

(51) Int. Cl. G16B 50/10; G16B 20/00; G16B 5/00; G06F 17/18

(52) U.S. Cl. CPC G16B 50/10; G06F 17/18; G16B 5/00; G16B 20/00

(57) ABSTRACT

Examining genomic information for a specific variance, while useful, provides limited information. Accordingly, systems and methods are provided to analyze a subject's genome against a background population. Outlier variances that are known ontological terms having at least a threshold strength-of-effect are then determined between each member. As a benefit, the subject's outlier variances, which may be further ranked in terms of relationship to a known phenotype, may be identified for an entirety or portion of

### FIELD OF THE DISCLOSURE

The present disclosure is generally directed towards the prediction of the phenotypic characteristics of an individual, of any species, based on their genome, which contains genetic outliers within a population.

### BACKGROUND

Analysis of the deoxyribonucleic acid (DNA) has provided a wealth of information related to a particular species, population, pool of organisms, and even specific individuals. Continuous discoveries lead to the ever-increasing knowledge of genetic data and how that data impact an individual's phenotype. For example, if a subject carries a mutation in the gene MCM6, encoding the lactase protein, it fails to produce sufficient lactase and exhibits lactose intolerance.

There are numerous examples of genotype-phenotype relationships in humans in diverse areas including e.g., medical/health, fitness/sport, aptitudes, nutrition, physical/physiological characteristics, psychological/mental etc. phenotypes spanning the areas of medical/health, fitness/sport, aptitudes, nutrition, physical/physiological characteristics, psychological/mental, etc. These encompass susceptibility/risk (e.g., to injury or disease), rates (e.g., faster recovery or adaptiveness, metabolism), quantity (e.g., bone density, cardiac capacity), or binary (e.g., absence of wisdom teeth or presence of an enzyme), reaction (e.g., to drugs, alcohol, caffeine), etc. Many have been discussed in scientific publications and include information regarding the underlying variant. These are the “known variants” and, although significant, represent only a miniscule fraction of a person's genetic variation. There are three billion base pairs, of which the order of 100,000 variants vary per person in protein-coding exons alone (which constitute ˜1.5% of the human genome), and there exist only ˜175,000 “known variants” across all people (e.g., number of entries in HGMD the Human Gene Mutation Database).

### SUMMARY

It is with respect to the above issues and other problems that the embodiments presented herein were contemplated. As one embodiment and a general introduction, a subject individual of a species is selected for analysis. Predicting the phenotypic characteristics of an individual through a causal analysis via molecular biology approaches may result in the identification of the genetic determinants responsible. Embodiments of the present disclosure are generally directed towards linking the variant with the processes of phenotypic outlier detection with the identification of the genetic cause.

A determination is made to identify which relevant variants the subject has in view of the background population of the species. One may wish to choose a generic human background with as much diversity as possible or a more targeted background. For example, lactose intolerance may be a rarity (e.g., statistical outlier) for certain human subjects in a generic population but not, for example, when compared to a population sample of Chinese individuals. Performing an analysis to determine lactose intolerance in a Chinese subject against a generic background may be of little interest; therefore, a population sample of Chinese individuals may be selected. In this way, however, a different population background sample may be selected in order to determine the variants the subject has with respect to that population. The variants may be further addressed in terms of relevancy, such as the identification of an association between a variant and a phenotype or genetic trait and the strength of such a predictor in indicating the trait.

SUPERFAMILY is a database comprising a library of Hidden Markov Models (HMMs) that represent the structural classification of proteins (SCOP). SUPERFAMILY provides a mapping of structural domains of proteins encoding DNA sequences, including genomes and other sequence sets and is accessible via the Internet address “http://supfam.org/SUPERFAMILY/” and is incorporated herein by reference. Additional information on HMM may be found in the paper “Predicting the Functional, Molecular, and Phenotypic Consequences of Amino Acid Substitutions using Hidden Markov Models.” Human mutation 34 (1), 57-65, Shihab, H. A., Gough, J., Cooper, D. N., Barker, G. L. A., Edwards, K. J., Day, I. N. M., Gaunt, T. (2012), which is incorporated herein in entirety by reference.

D²P² is a database comprising records of pre-computed disordered predictions from completely sequenced genomes. D²P² is accessible via the Internet address “http://d2p2.pro/” and is incorporated herein in entirety by reference.

FATHMM (“Functional Analysis through Hidden Markov Models”) is a resource for predicting the function consequences of both coding variants (e.g., non-synonymous single nucleotide variants (nsSNVs)), and non-coding variants. FATHMM is accessible via the Internet address “http://fathmm.biocompute.org.uk/” and is incorporated herein in entirety by reference.

dcGO is a database of domain-centric ontologies on functions, phenotypes, diseases, and additional related information. dcGO is accessible via the Internet address “http://supfam.org/SUPERFAMILY/dcGO/” and is incorporated herein in entirety by reference.

Spectral clustering provides a grouping based on a number of dimensions. Spectral clustering, when used in image analysis segments an image into logical associations, such as areas of an image, using vectors of color/brightness, etc. (e.g., in a landscape: sky, sun, tree, field, lake). In terms of a human, to which the embodiments herein relate, a phenotype such as eye color should segment the population into the basic eye colors using vectors of alleles: brown, green, blue, hazel, or grey. Quantitative scores are output for each individual person/pixel based on the difference they exhibit from their local neighborhood as opposed to a global one. A global comparison will simply rank the person with the bluest eyes as the furthest outlier, whereas spectral clustering will identify the one rare person who has yellow eyes but lies in the center of the eye color landscape.

In another embodiment, outlier detection is provided by intrinsic dimensionality. Intrinsic dimensionality is used for local outlier detection as a method of reducing the genetic variants down to a combination of those variants that are most relevant to predicting the phenotype in question. Where spectral clustering uses all dimensions, the intrinsic dimensionality describes how many variables are needed to represent the phenotype. Both methods have been previously used in computer vision. A more detailed definition and an example of usage of intrinsic dimensionality may be found in, “Intrinsic Dimensional Outlier Detection in High-Dimensional Data,” Jonathan von Brünken, Michael E. Houle, and Arthur Zimek, National Institute of Informatics, which is incorporated herein in entirety by reference and available at the Internet address “http://www.nii.ac.jp/TechReports/15-003E.pdf.”

In one embodiment, a method is disclosed, comprising: accessing, by a processor, genomic data of a number of individual organisms comprising a subject and a background population; identifying, by the processor, a number of variant sites in the genomic data; for one of the number of variant sites: retrieving, by the processor, from a first data repository by the processor, a strength-of-effect associated with the one of the number of variant sites; retrieving, by the processor, from a second data repository by the processor, a degree of association with an ontological term associated with the one of the number of variant sites; and determining, by the processor, that one of the number of phenotypes is relevant upon the strength-of-effect in combination with a degree of association with the ontological term being above a threshold amount; comparing, by the processor, a degree of match between a number of data pairs, each data pair comprising one datum of genomic data for the relevant phenotype from substantially each combination formed by two of the number of individuals; and generating, by the processor, a report for each of the pairs.

In another embodiment, a system is disclosed comprising: a communication interface; a processor; and wherein the processor performs accessing, by a processor, genomic data of a number of individual organisms comprising a subject and a background population; identifying, by the processor, a number of variant sites in the genomic data; for one of the number of variant sites: retrieving, by the processor, from a first data repository by the processor, a strength-of-effect associated with the one of the number of variant sites; retrieving, by the processor, from a second data repository by the processor, a degree of association with an ontological term associated with the one of the number of variant sites; and determining, by the processor, that one of the number of phenotypes is relevant upon the strength-of-effect in combination with a degree of association with the ontological term being above a threshold amount; comparing, by the processor, a degree of match between a number of data pairs, each data pair comprising one datum of genomic data for the relevant phenotype from substantially each combination formed by two of the number of individuals; and generating, by the processor, a report for each of the pairs.

It should be appreciated that the processor may comprise one or more of a microprocessor, one or more processing cores, one or more servers/blades, distributed processor (e.g., “cloud”), and/or other computing devices operable to carry out computations, programmed method steps and/or may be implemented to perform method steps disclosed herein. The processor may be associated with a communication interface, such as a network interface operable to access data locally (e.g., a bus, backplane, etc.), remotely (e.g., intranet, Internet, etc.), and/or other communication interfaces. The processor may further comprise human input-output signals to enable human input devices (e.g., mouse, trackpad, keyboard, etc.) to provide instructions to the processor and/or human output devices (e.g., screen, terminal, monitor, printer, etc.) to provide human-readable output. The processor may access a data repository for the reading/writing of data and, optionally retrieval of computer instructions that when read by the processor cause the processor to perform the method steps disclosed herein. The data repository may be a storage device accessed locally (e.g., internal drive, registers, memory chips, etc.) to the processor and/or remotely (e.g., external drive, networked storage device, Intranet/“cloud” storage, etc.). As a result, a processor may access programming instructions and data to perform certain method steps disclosed herein and output the results to a storage device and/or output device for production of a human-readable output.

The phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium,” as used herein, refers to any tangible storage that participates in providing instructions to a processor for execution. Such a medium may take many forms, including, but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, an EPROM, a FLASH-EPROM, a solid-state medium like a memory card, any other memory chip or cartridge, or any other medium from which a computer can read. When the computer-readable media is configured as a database, it is to be understood that the database may be any type of database, such as relational, hierarchical, object-oriented, and/or the like. Accordingly, the disclosure is considered to include a tangible storage medium and prior art-recognized equivalents and successor media, in which the software implementations of the present disclosure are stored.

The terms “determine,” “calculate,” and “compute,” and variations thereof, as used herein, are used interchangeably and include any type of methodology, process, mathematical operation, or technique.

The term “module,” as used herein, refers to any known or later-developed hardware, software, firmware, artificial intelligence, fuzzy logic, or combination of hardware and software that is capable of performing the functionality associated with that element. Also, while the disclosure is described in terms of exemplary embodiments, it should be appreciated that other aspects of the disclosure can be separately claimed.

### BRIEF DESCRIPTION OF THE DRAWINGS

The present disclosure is described in conjunction with the appended figures:

FIGS. 1A-1E depict a process in accordance with embodiments of the present disclosure;

FIG. 2 depicts a report in accordance with embodiments of the present disclosure;

FIGS. 3A-3D depict a first table in accordance with embodiments of the present disclosure;

FIG. 4 depicts a second table in accordance with embodiments of the present disclosure;

FIG. 5 depicts a system in accordance with embodiments of the present disclosure; and

FIG. 6 depicts a data processing model in accordance with embodiments of the present disclosure.

### DETAILED DESCRIPTION

The ensuing description provides embodiments only and is not intended to limit the scope, applicability, or configuration of the claims. Rather, the ensuing description will provide those skilled in the art with an enabling description for implementing the embodiments. It will be understood that various changes may be made in the function and arrangement of elements without departing from the spirit and scope of the appended claims.

Any reference in the description comprising an element number, without a subelement identifier when a subelement identifier exists in the figures, when used in the plural, is intended to reference any two or more elements with a like element number. When such a reference is made in the singular form, it is intended to reference one of the elements with the like element number without limitation to a specific one of the elements. Any explicit usage herein to the contrary or providing further qualification or identification shall take precedence.

Certain embodiments, including systems and methods, disclosed herein may be embodied as software, hardware, software-hardware combinations (firmware), modules, and/or associated analysis hardware. However, to avoid unnecessarily obscuring the present disclosure, the following description omits well-known structures, components, and devices that may be shown in block diagram form and are well known or are otherwise summarized.

For purposes of explanation, numerous details are set forth in order to provide a thorough understanding of the present disclosure. It should be appreciated, however, that the present disclosure may be practiced in a variety of ways beyond the specific details set forth herein.

The prior art associates phenotypes to genotypes via genome-wide association studies (GWAS). GWAS target a specific phenotype, such as a human disease. GWAS typically collect genotype data from a large pool of individuals, commonly 1000-10,000, some of which will and will not have the phenotype selected. Statistically significant correlations are then sought between a genetic feature and the disease.

Despite the successes of GWAS, limitations exist. One limitation is the massive scale of the required genotyping. Another limitation is the insufficiency of statistical power to associate combinations of genotypes with phenotypes. Still another limitation is that the associations of the genotypes are often non-causal correlations. Additionally, cohort size on statistical power results in only finding significant associations with common variants. The embodiments provided herein address these and other limitations and advance the state of the art of, at least, genotype-phenotype association determination. Benefits of certain embodiments provided herein allow causal relationships to be determined, require a smaller number of background samples compared to GWAS, provide associations for common or rare variants, detect combinatorial genotypes, and may be more reliably predictive.

In one embodiment, a single subject, such as a human, provides their genetic data as an input. The data may be complete or comprise only a portion of their complete genetic data. The data is then used to predict aspects of their phenotype that are unusual or outstanding when compared to a population, which may be a general population (e.g., humans) or more targeted (e.g., humans of Korean ancestry).

In one embodiment, systems and methods are described directed to the prediction of attributes of a variant. In another embodiment, multiple cross-referenced data sets are used and, when taken together, provide a statement about a variant that is not known in the body of scientific literature. Predictions may then be made about more variants than are “known.” In a further embodiment, predictions may be made on rare variants, even to the point of being unique to one person. In another embodiment, predictions may be made on the combinations of multiple variants (i.e., if a person has variant “a” and “b,” “a” and “b” but not “c,” or not “d,” etc.).

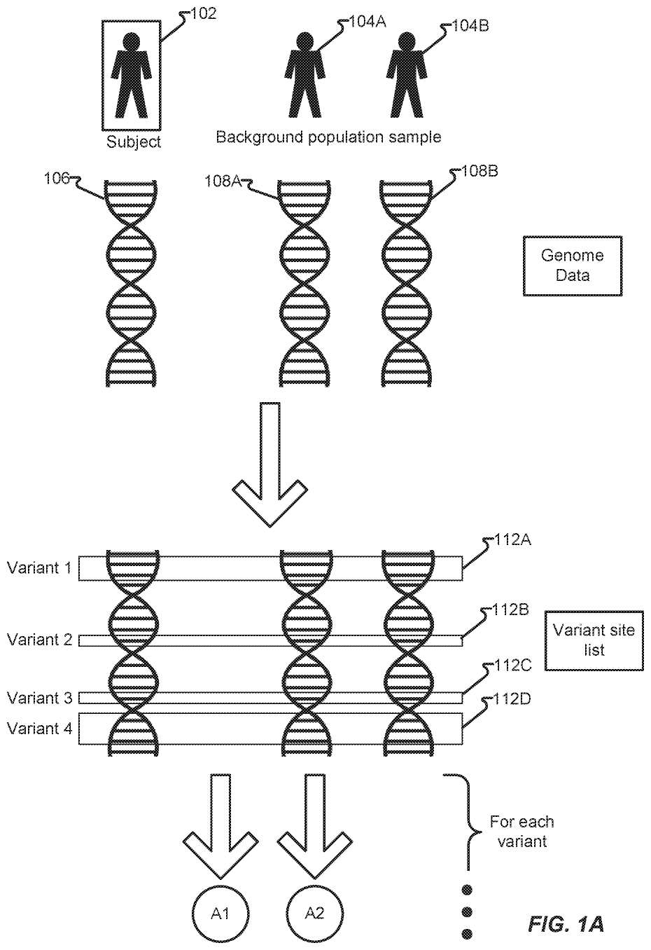

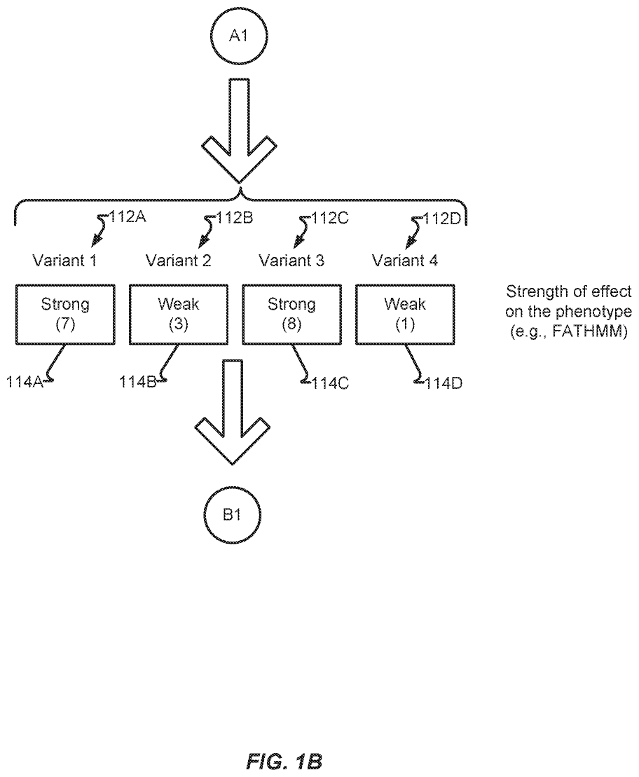

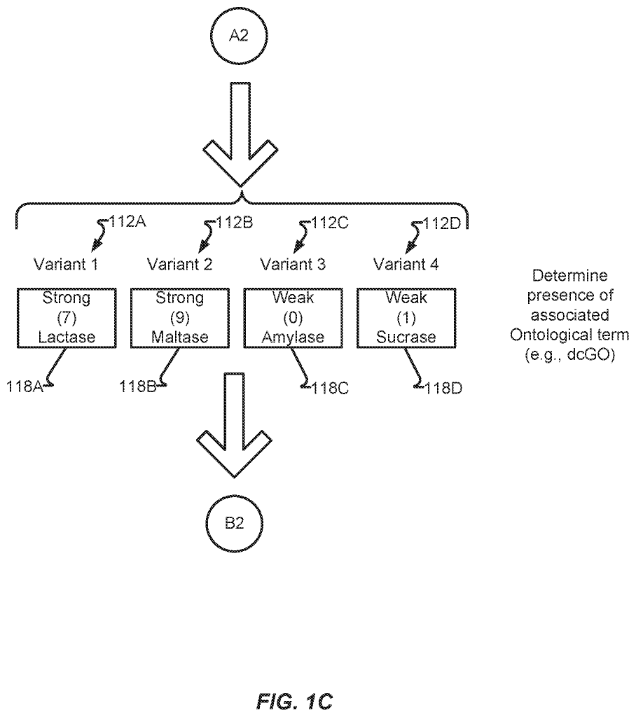

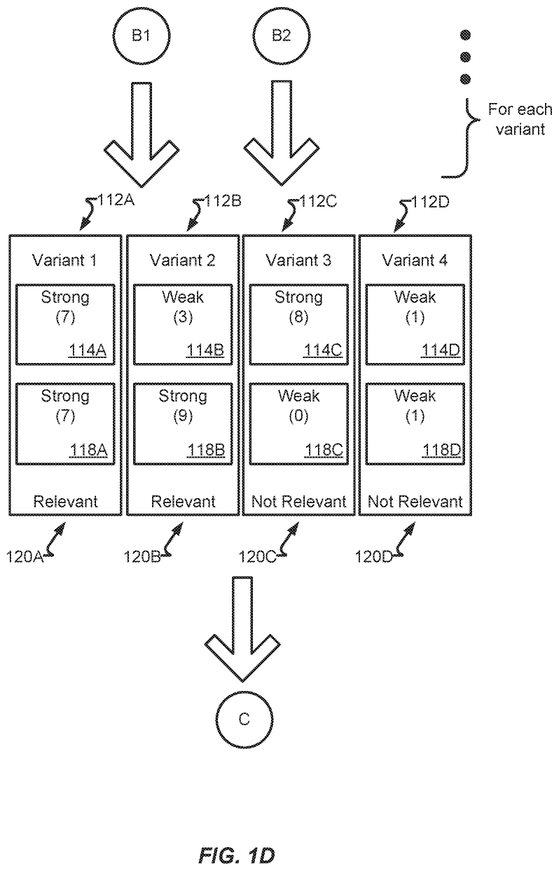

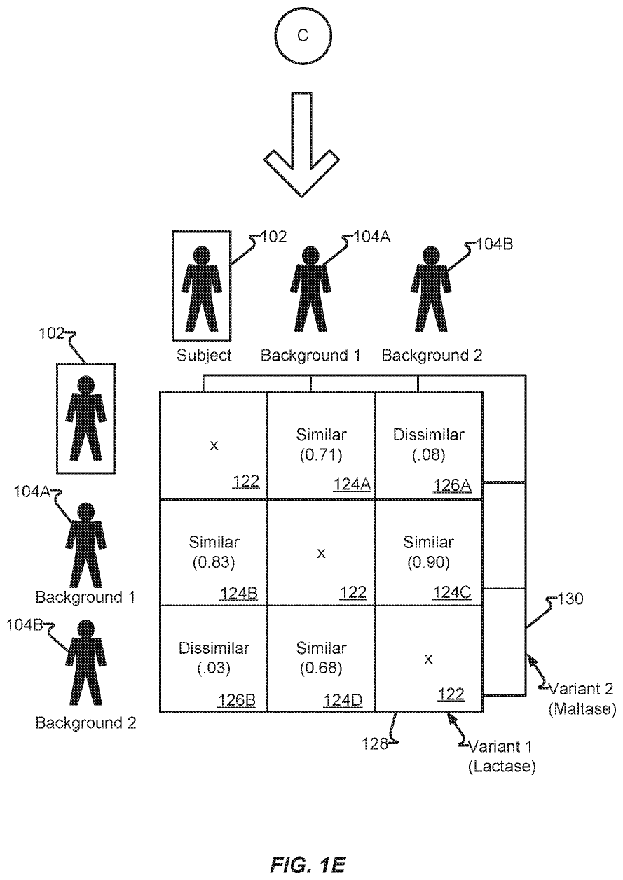

FIGS. 1A-1E depicts a process in accordance with embodiments of the present disclosure. In one embodiment, a variant-effect predictor tool and a function-effect predictor tool are each provided with genotype data using protein domains of a background population and a subject. The results are combined, such as by utilizing spectral clustering, and optionally configured based upon selected parameters. The results may then be ranked and prioritized, such as to provide outlier details for a given phenotype, which may include a determination of a most extreme phenotype for a subject.

In one embodiment, genomic data 106, 108A-108B are gathered from a pool of organisms. The genotype data may be a collection of single nucleotide polymorphisms (SNPs), an exome, or a full genome sequence. Genomic data comprise genomic data 106 from at least one subject 102 and genomic data 108 from background population 104A-104B. Background population 104 is preferably selected to comprise a statistically significant number of members. Subject 102 may be, or not be, determined to be an outlier in the background population within a previously determined significance, as may be determined by an objective. For example, subject 102 may be a known outlier with respect to one phenotype for a background and a determination is desired as to other phenotypes for which the subject may, or may not be, an outlier. In another example, subject 102 has unknown outlier status. In yet another embodiment, background population 104 is selected to be arbitrarily large (e.g., 1000 members or more).

In another embodiment, the selection of substantially all members of background population 104 is selected in accordance with a desired determination against a desired population. For example, subject 102 has a number of known general characteristics (e.g., gender, ethnicity, etc.) and/or known specific characteristics (e.g., phenotypes). Background population 104 may then be selected having the same, different, or a mix of similar, general, and/or specific characteristics. For example, subject 102 is a Korean male and, if it is desired to determine the significant genetic outliers of subject 102 among all Korean males, then background population 104 preferably comprises only Korean males. However, if an object of a particular analysis is to determine the significant outliers of subject 102 compared to all humans, then background population 104 preferably comprises a representative sample of the entire human population. In another embodiment, background population 104 is selected from a public genome database (e.g., “23andme”). Genomic data 106, 108 may be complete or contain one or more portions of a complete genome.

In another embodiment, genomic data 106, 108 is then converted into a list of variant sites relative to a background population and processed into variant sites 112A-112D in Variant Call Format (VCF). VCF provides one way to store the genome of an individual organism in terms of the difference to a reference genome, or all differences between multiple individuals in a cohort and each other.

Variant sites 112 are then processed to predict the molecular entities of which the variant is a part, controls, or encodes. In one embodiment, processing of variant sites 112 is performed using hidden Markov Model (HMM) predictions. In another embodiment, SUPERFAMILY and D²P² are applied to genome 106 of subject 102. In another embodiment, variants in non-coding regions are associated with proteins via their proximity to genes or by known and predicted features such as regulatory elements upstream and downstream of the genes. In another embodiment, variants in non-coding regions are associated with proteins via associations using high throughput datasets such as CAGE, DNAase hypersensitive sites (DHS), RNA-seq, and replication timing or chromatin exposure.

Variant sites 112 are illustrated as single 112B, 112C or neighboring multiple 112A, 112D bases as a matter of convenience. Variant sites 112 may include non-neighboring bases, alternatives (e.g., A and B or C), exclusions (e.g., A and not D), and/or other variants that are or may become known. For each variant site 112, a strength effect is determined and ontological term identification is performed (see, FIGS. 1B and 1C, respectively).

FIG. 1B illustrates one embodiment wherein a strength-of-effect is determined for a phenotype for each variant site 112. In one embodiment, the variant-effect predictor tool comprises the FATHMM database. It should be appreciated that other strength-of-effect determinations may be made, such as by accessing other data repositories. It should also be noted that FATHMM may be untilized to make variant-effect predictions for both coding and non-coding variants and in the context of either somatic or germ-line mutations.

In one embodiment, each variant site 112 is compared to an entry in a database, or variant-effect predictor tool, accesses a causal relationship indicator, such as score 114A-114D. Score 114 may be a raw score, such as indicating the position of a particular variant (e.g., variant 112A) on a known scale. In another embodiment, other indicators or labels may be utilized, such as to readily identify variants that do and/or do not have a significant strength-of-effect to a phenotype (e.g., “strong,” “weak,” etc.) or as a matter of convenience. The resulting strength-of effect is aggregated with ontological term identification (see, FIG. 1D).

FIG. 1C illustrates one embodiment wherein a determination is made as to a variant (e.g., ones of variants 112) having an associated ontological term 118A-118D. For example, variant 1 (112A) may be associated with ontological term 118A (lactase). It should be appreciated that, due to variations within a single ontological term, the determination may be a score and a binary “yes/no” determination may indicate a determination score above/below a threshold value on a known scale. The resulting ontological term determination is then aggregated with strength-of-effect data (see, FIG. 1D).

FIG. 1D illustrates one embodiment wherein a relevancy is determined as a combined strength-of-effect on a phenotype (see, FIG. 1B) and ontological term determination (see, FIG. 1C) to provide a single score indicating that at least one variant sites 112 is both a significant predictors and the significant predictor is of a known ontological term. Scores of one or both of strength-of-effect data and ontological term determination data may be weighted, such as to reflect a combination different from the sum of the individual data sources. In another embodiment, the scores may then be determined to be relevant 120A, 120B or not 120C, 120D, such as when the combined scores are above a previously determined threshold value or formula (e.g., top 0.5%, etc.). Variant sites 112 determined to be relevant data are then processed further (see, FIG. 1E).

FIG. 1E illustrates one embodiment, wherein distances are determined for subject 102 and background population 104 for each relevant variant (e.g., variant 1 (128), variant 2 (130), etc.). In one embodiment, matrices are formed for variant 128, 130. Cells 122 indicate same-to-same comparisons. Cells 124A-124D, 126A-126B may be scored to indicate a distance (or similarity, degree of match, etc.) between that particular organism (e.g., one of subject 102, background 104, etc.) and another organism (e.g., a different one of subject 102, background 104, etc.). Therefore, in one embodiment, each organism can be compared to each other organism. The distance between subject 102 and background 104 may then be indicated as an outlier, or not, for a particular variant.

In another embodiment, the score may be determined utilizing term frequency-inverse document frequency (TF-IDF).

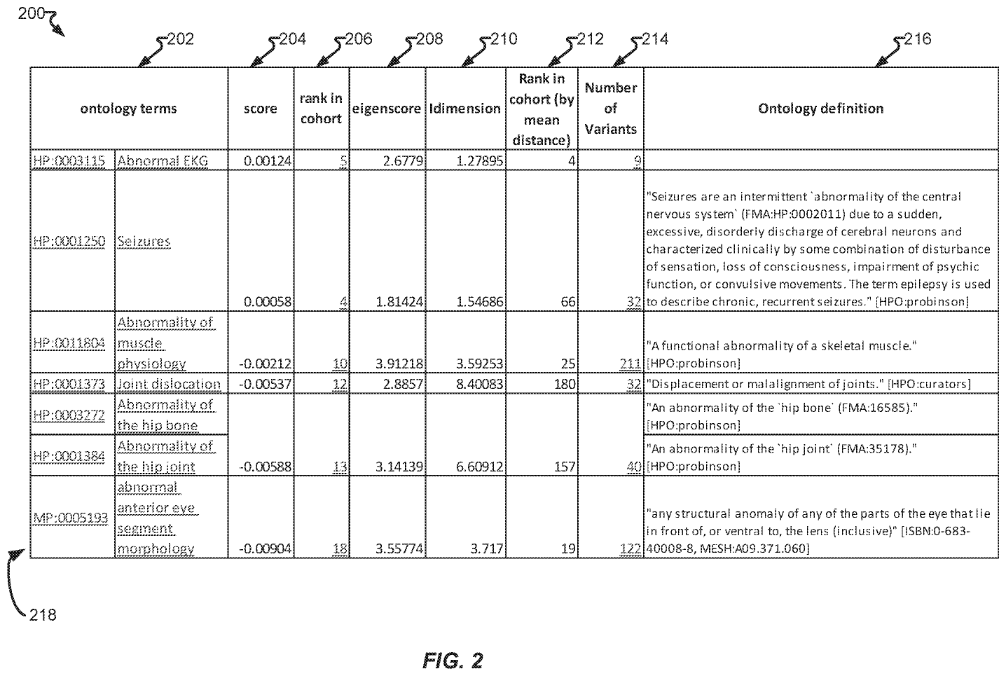

FIG. 2 depicts report 200 in accordance with embodiments of the present disclosure. Report 200 may be provided for an individual, such as subject 102 and may further provide variants, in rows 218 of table 200, having both an ontological term and a strength-of-effect above a threshold value and has been jointly determined, for one or more collections of variants relevant to the ontological term (e.g., number of variants 214 for a respective row 218), to have statistical differences from a background population. In one embodiment, three variants are individually low-scoring due to commonality within the population or at least a significant portion of the population (e.g., approximately half). However, if the combination of the three variants is rare, then such a combination may be highly significant. In another embodiment, spectral clustering is utilized to identify outliers comprising two or more variants.

Accordingly, report 200 may then comprise one or more rows 218 of ontological terms 202 (e.g., abnormal EKG), which in turn may comprise an identifier (e.g., “HP:0003115”), such as that taken from an ontology database such as “Human Phenotype” accessible via the Internet address http://human-phenotype-ontology.github.io, “Mammalian Phenotype,” accessible via the Internet http://www.informatics.jax.org/searches/MP_form.shtml, “Gene Ontology,” accessible via the Internet address http://amigo.geneontology.org, “Drugbank,” accessible via the Internet address “http://www.drugbank.ca,” and/or “dcGO” (see above) each of which is incorporated herein in entirety by reference, and/or other data repository of ontological terms.

In one embodiment, score 204 combines rank 206 and eigenscore 208. In another embodiment, score 204, combines rank 206 and idimension 210, in place of eigenscore 208. Score may be determined by formula:

$$\begin{matrix}score = {( x * y * z )}^{\frac{1}{3}} & ( i ) \\ wherein \text{:} x = \frac{{r}^{1 + \frac{rank}{n}}}{r} & ( ii ) \\ wherein \text{:} y = \frac{eigenscore}{s} & ( iii ) \\ wherein \text{:} z = \frac{eigenscore - m 1}{m 2} & ( iv ) \\ wherein \text{:} r = {e}^{150} & ( v )\end{matrix}$$

wherein m1 is the minimum score of any individual in the cohort;

wherein m2 is the maximum score of any individual in the cohort;

wherein n is the number of individuals in the cohort;

wherein s is the sum of all Eigenscores in the cohort; and

wherein rank is rank within the background population.

In another embodiment, rank (e.g., rank in cohort 206) provides a ranking within the background population by score 204; eigenscore is eigenscore 208 indicating the raw score from spectral clustering; idimension 210, comprise the raw score from intrinsic dimensionality; rank in cohort by mean distance 212, comprise the rank within the background population by idimension 210; number of variants 214, comprise the number of variants that are relevant as determined by ontology association (e.g., as indicated by dcGO), and ontology definition 216, comprising the formal definition provided by the source ontology database.

As used herein, cohort comprises the participants (e.g., the background individuals, such as one or more background 104 and one or more subject 102) and provides the background plus the individual(s) in question. “Mean ave. distance” may be substituted for “Eigenscore,” such as in score column 204, and optionally in formula (i), to report by intrinsic dimensionality instead of spectral clustering.

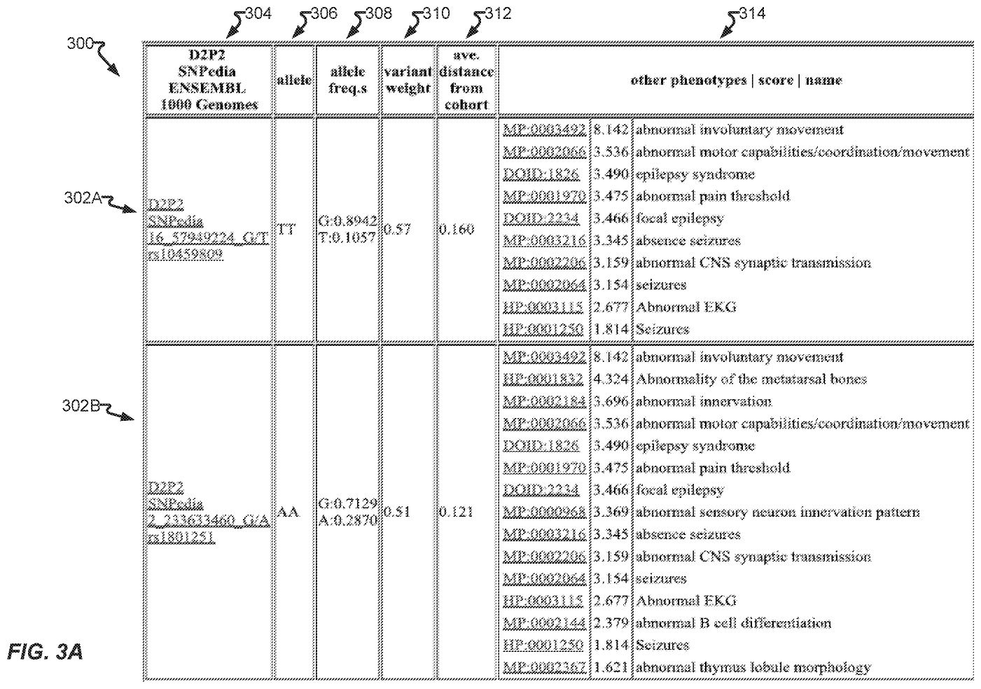

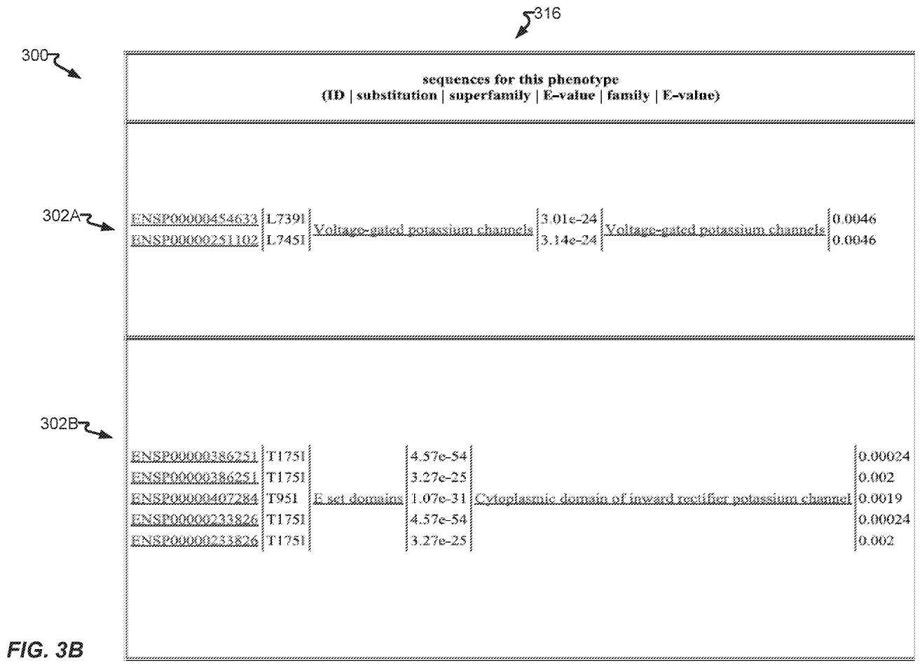

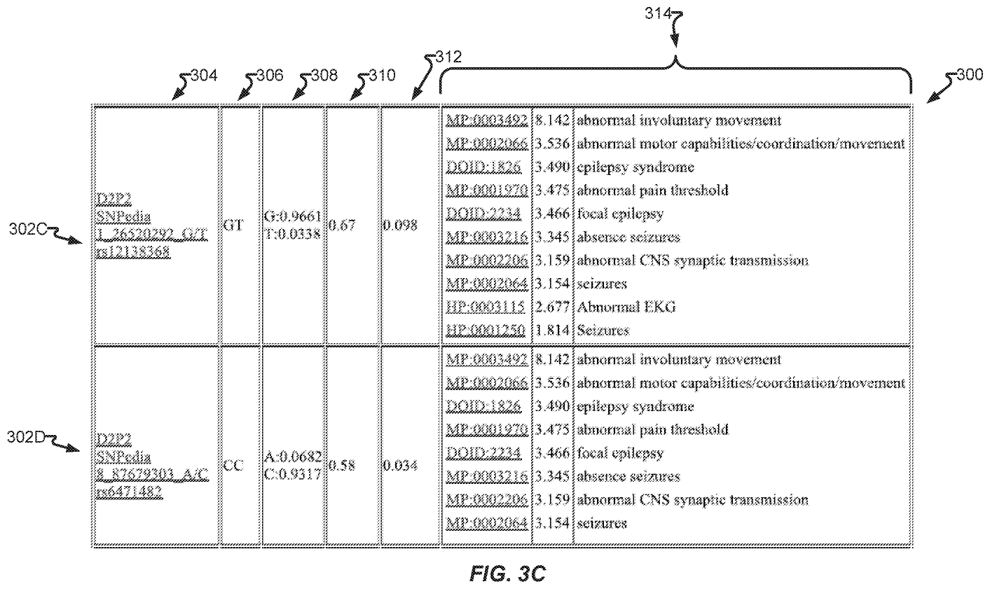

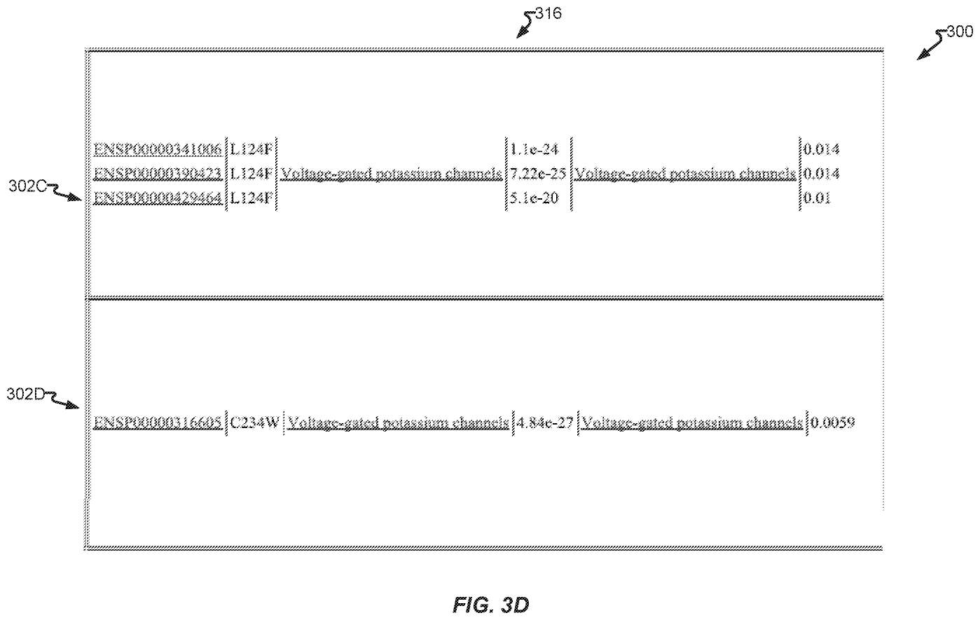

FIGS. 3A-3D depict table 300 in accordance with embodiments of the present disclosure. In one embodiment, table 300 depicts rows 302A-302D and columns 304, 306, 308, 310, 312, 314, and 316. In one embodiment, table 300 presents the causal biological reason for the phenotype being an outlier. Table 300 corresponds to one row of table 200, i.e. column 214 lists only the number of variants whereas table 300 contains one row for each of those variants, e.g. for ‘abnormal EKG’ there are 9 variants and thus the complete table 300 would contain 9 rows, but only the first two are shown for illustration. Column 304 shows the genomic coordinates of the variant, column 306 shows the alleles of the subject, column 308 shows allele frequencies in the background, column 310 shows variant weight or consequence score (e.g., from FATHMM), column 312 shows average distance from cohort using the distance matrix (i.e., the contribution of that variant taken alone), column 314 identifies other phenotypes connected to the same variant, which may further include a score and name. Column 316 shows information about the variant (e.g., a corresponding one of row 302) namely sequences affected by the variant in a particular row 302 and may further comprise an identifier (ID), amino acid substitution, superfamily, e-value, family, and family e-value.

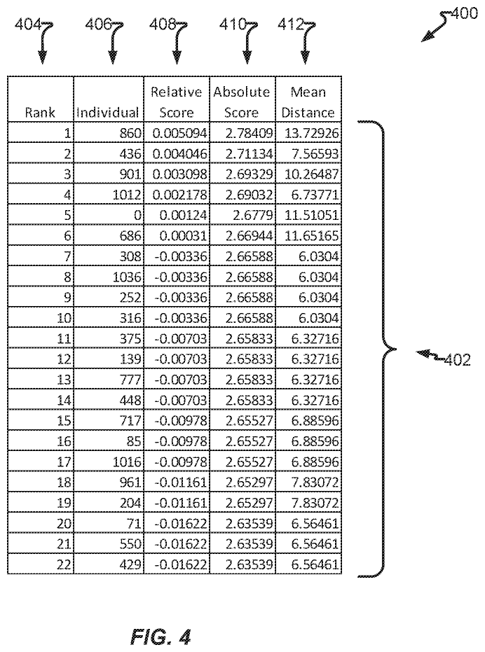

FIG. 4 depicts table 400 in accordance with embodiments of the present disclosure. In one embodiment, table 400 comprises a number of rows 402 and columns 404, 406, 408, 410, and 412. In one embodiment, table 400 can be used to see the distribution of scores amongst the background and to compare spectral clustering scores to intrinsic disorder scores. In one embodiment, column 404 provides a rank, for example, rank 206 of table 200 for one individual; entries in column 406 identify the individuals within row 402, such as by a numeric identifier. In another embodiment, an individual number of zero is associated with the subject such as subject 102; or, if more than one, one of a number of subjects 102. In another embodiment, column 408 shows a score relative to one individual (e.g. the subject in question in table 200), column 410 shows an absolute eigenscore as per column 208 for one individual, and column 412 provides the alternative score from intrinsic dimensionality as per column 210 for one individual.

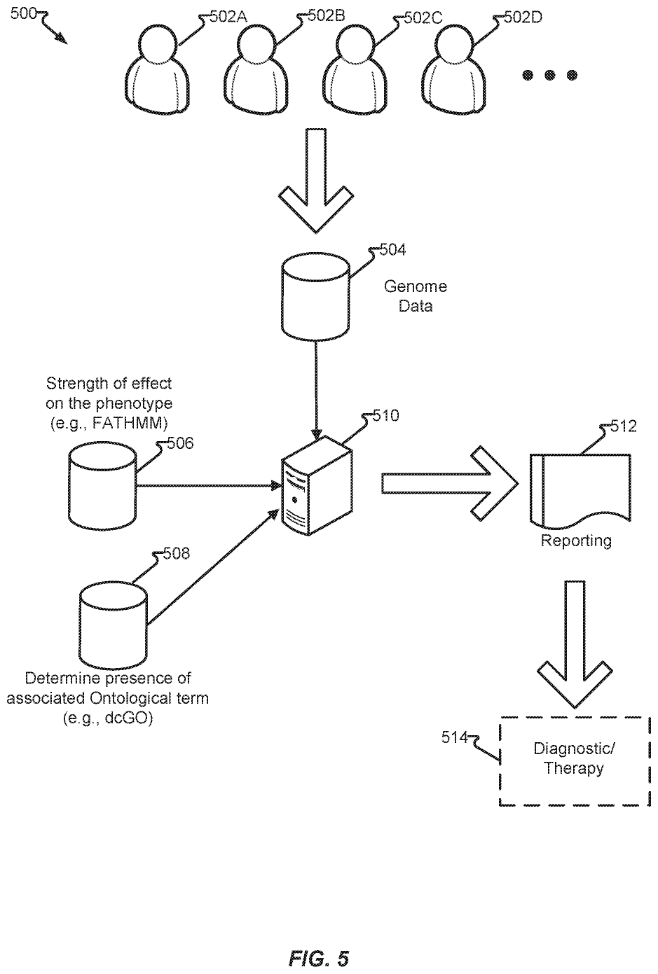

FIG. 5 depicts system 500 in accordance with embodiments of the present disclosure. In one embodiment, organisms 502A-502D provide genomic data 504, such as from individuals identified in column 406 (See, FIG. 4). At least one, but less than all organisms 502, is identified as a subject organism(s). Processor 510 accesses, for variant sites in genomic data 504, strength-of-effect data 506 and ontological term data 508 to determine significant outliers for a subject. Processor 510 may then provide report 512, such as ranking significant outliers, which may optionally be provided for diagnostic and/or therapeutic treatment 514.

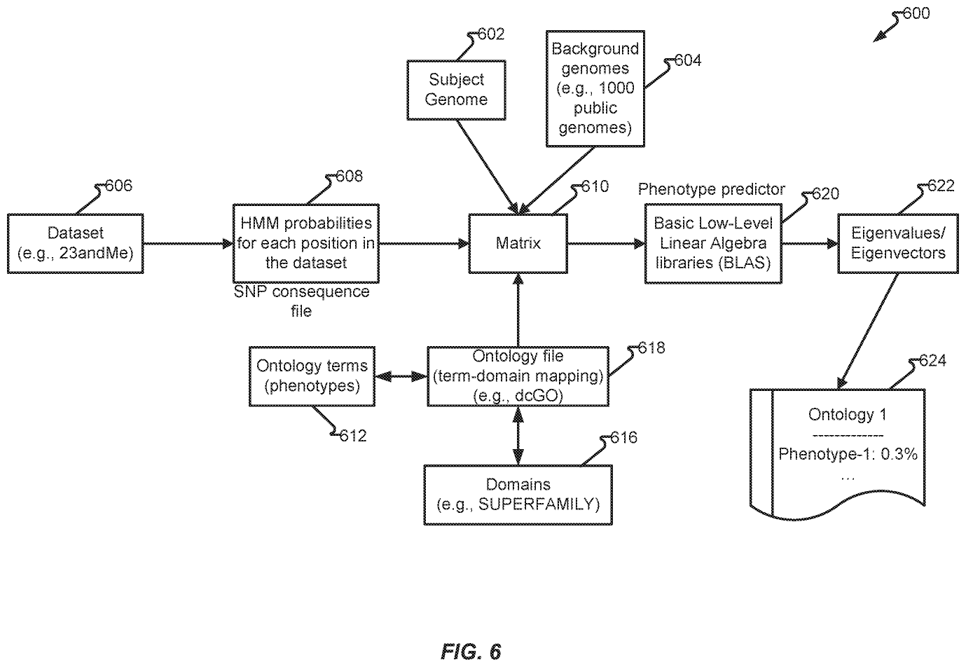

FIG. 6 depicts data processing model 600 in accordance with embodiments of the present disclosure. In one embodiment, subject genome 602 and background genomes 604 are combined in a matrix for each phenotype to determine distances between subject genome 602 and background genomes 604 for such phenotypes.

In one embodiment, a distance within matrix 610 is obtained between all participants (the combination of subject 102 and background population 104) by adding FATHMM distance contributions from each SNP mapped to the ontology term.

Next, the Gaussian kernel is determined by the formula:

$$G ( x , σ ) = exp ( - \frac{{x}^{2}}{{σ}^{2}} ) \qquad ( iv )$$

and is applied to transform the distance matrix into a similarity matrix, where

σ=\sqrt{max(x²)}. (iiv)

Then a modified version of spectral clustering is performed:

From the similarity matrix, the Laplacian (defined as Degree-Similarity) is obtained to solve the eigenproblem.

The second smallest eigenvector is used to partition the dataset into clusters. In another embodiment, a modified version of the elbow method is used to choose first N smallest eigenvectors to use for the clustering. Then the chosen eigenvectors are used to partition into clusters via standard K-means clustering.

Normally, when used for image partitioning, spectral clustering uses the first N eigenvectors to partition the image into N clusters. However, for genomic data, such as by utilization of multidimensional scaling, use of only the first eigenvector may be employed to partition the genomic data into more than two clusters. Two clusters may be particularly relevant in implementations partitioning on one SNP having all three options in common.

The number of clusters N may be chosen as described by Pham, Duc Truong, Stefan S. Dimov, and C. D. Nguyen. “Selection of K in K-means clustering.” Proceedings of the Institution of Mechanical Engineers, Part C: Journal of Mechanical Engineering Science 219.1 (2005): 103-119, which is incorporated herein in entirety by reference.

Next, scores for each participant are calculated as follows:

The combination of the Euclidean distance from the center of the local cluster and the distance of the cluster from the global center.

A score is determined by the formula:

Score=Local_score+μ·Global_score (iix)

And the correction for cluster size is determined by the formula:

$$μ = \frac{{e}^{( γ \cdot \frac{cohor t \_ size - cluster \_ size}{cohort \_ size} )} - 1}{{e}^{γ} - 1} \qquad ( ix )$$

Score comprises a distance between one of the genomic data and the rest of the background, as in column 208 of table 200. Local_score comprises an average Euclidian distance from one of the number of individual organisms to the cluster comprising each of the number of individual organisms in the same cluster as the subject.

Global_score comprises an average Euclidian distance for ones of the number of organisms within a cluster to all other of the number of organisms within the cohort.

Cohort_size comprises the number of the number of individual organisms in the cohort, which is the same as the number of rows as would be in the complete table, part of which is shown in FIG. 4.

Cluster size comprises the number of the number of individual organisms from the cohort in the cluster.

In another embodiment, dataset 606 is provided to SNP consequence file 608, such as comprising HMM probabilities. In another embodiment, ontological terms 612 are mapped via ontology file 618 (e.g., dcGO) to domains 616 (e.g., SUPERFAMILY). Ontology file 618 and SNP consequence file 608 may then be further utilized to determine distances within matrix 610.

In another embodiment, phenotype predictor 620, such as one using basic low-level linear algebra libraries (BLAS), produces eigenvalues and eigenvectors 622. As a result, report 624 may be produced, which may report the degree that subject genome 602 may be from background genomes 604 for a particular ontological term.

In another embodiment, variants may be prioritized, such as by prioritizing variants in the genome for further investigation. For example, in a cohort of patients with a known disease, variants may be prioritized on the basis of how common they are in the cohort and how rare in the background, such as in GWAS studies. In another embodiment, genes may be prioritized for further study in a manner similar to variant prioritization. It is possible that no two members of the cohort share the same variant, but they may all have different variants in a common gene, suggesting that gene should have a high priority.

In another embodiment, phenotype prioritization is provided, comprising prioritizing ontological terms for further investigation. An example would be for one individual, given all of their phenotype predictions may have priority given to those phenotypes for which that individual is a more or most of an outlier as compared to the background according to the genetic variation. There may be multiple genes/variants that contribute to the phenotype in one individual. Alternatively for a cohort with something in common, (e.g., a common disease), prioritization of phenotypes might identify which ontological terms have the most outlying genetic variation in associated genes within the cohort relative to a background.

In another embodiment, phenotype prioritization score comprises a score for each phenotype for an individual. The score may be the result of a function that takes into consideration both an absolute score, such as from spectral clustering or intrinsic dimensionality, and the distribution of such scores in the background, such as via ranking. To prioritize ontological terms for further investigation from an individual, the resulting prioritization score, such as obtained via formula (i), may be used to rank the phenotypes on priority. Phenotype prioritization may then identify the characteristics of an individual that are most genetically outlying with respect to the background. In a further embodiment, the result can be extended to phenotype prioritization of a cohort by choosing a threshold and, for each phenotype term, counting the number of members of the cohort who have an above-threshold score.

In another embodiment, gene and variant prioritization may be provided without phenotype prediction by examining the frequency of variation within a cohort versus a background. Another embodiment combines a variant score (e.g. from FATHMM) with the frequency. In such embodiments, gene and variant prioritization are taken from the phenotype prediction.

In another embodiment, there is no pre-conceived relevance of terms. For an individual, the variants are prioritized as a combination of the phenotype prioritization score and the variant prioritization score (e.g., the top priority variant will be that within the list of variants associated with the top-priority phenotype term), that has the top variant priority within that group. For gene prioritization the individual variant prioritization scores can be combined (e.g., via summation for each gene). For a cohort phenotype-based variant/gene prioritization may be similar to the forgoing but utilizing the cohort phenotype ranking instead of the individual. In another embodiment there is a pre-conceived notion of which phenotypes are of interest (e.g., in a cohort of neonatal fatalities), and the phenotype term (e.g., “neonatal lethal”) may be used. A more general embodiments list terms associated with neonatal lethality (e.g., “developmental disorder,” “abnormality of the placenta,”) may be used. Variant or gene prioritization may be restricted to those ontological terms which may have some connection with the pre-conceived notion of relevance.

In yet another embodiment, a treatment may be applied to subject 102 based upon the results produced by embodiments disclosed herein. The treatment may be the application or avoidance of a drug, genetic treatment, surgery, exercise, physical/occupational therapy, diet, environmental, and/or other medical, pharmacological, genetic, or lifestyle treatment. Treatment may further comprise inclusion of atypical examinations in terms of type, frequency, or time of examination based upon, for example, a perceived susceptibility for a potential phenotype.

In the foregoing description, for the purposes of illustration, methods were described in a particular order. It should be appreciated that in alternate embodiments, the methods may be performed in a different order than that described. It should also be appreciated that the methods described above may be performed by hardware components or may be embodied in sequences of machine-executable instructions, which may be used to cause a machine, such as a general-purpose or special-purpose processor (GPU or CPU), or logic circuits programmed with the instructions to perform the methods (FPGA). These machine-executable instructions may be stored on one or more machine-readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Specific details were given in the description to provide a thorough understanding of the embodiments. However, it will be understood by one of ordinary skill in the art that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Also, it is noted that the embodiments were described as a process, which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine-readable medium, such as a storage medium. A processor(s) may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

While illustrative embodiments of the disclosure have been described in detail herein, it is to be understood that the inventive concepts may be otherwise variously embodied and employed, and that the appended claims are intended to be construed to include such variations, except as limited by the prior art.

What is claimed is:

1. A method, comprising:
accessing, by a processor, genomic data of a number of individual organisms comprising a subject and a background population;
identifying, by the processor, a number of variant sites in the genomic data;
for one of the number of variant sites:
retrieving, by the processor, from a first data repository by the processor, a strength-of-effect associated with the one of the number of variant sites;
retrieving, by the processor, from a second data repository by the processor, a degree of association with an ontological term associated with the one of the number of variant sites; and
determining, by the processor, that one of the number of phenotypes is relevant upon the strength-of-effect in combination with a degree of association with the ontological term being above a threshold amount;
comparing, by the processor, a degree of match between a number of data pairs, each data pair comprising one datum of genomic data for the relevant phenotype from substantially each combination formed by two of the number of individuals; and
generating, by the processor, a report for each of the pairs.

2. The method of claim 1, wherein the subject comprises a plurality of subjects.

3. The method of claim 1, wherein the comparison is weighted based upon an associated repository entry for known consequences of variants.

4. The method of claim 1, wherein the report further comprises an indicator of statistical significance for the subject with respect to ones of the number of phenotypes.

5. The method of claim 1, wherein the step of generating the report further comprises generating, by the processor, a spectral clustering report.

6. The method of claim 1, wherein the step of generating the report further comprises generating, by the processor, an intrinsic dimensionality report.

7. The method of claim 1, wherein for ones of each of the number of phenotypes performing the comparing step is performed, by the processor, upon the processor determining that the phenotype, determined as the combined strength-of-effect and degree of association with an ontological term, is above the threshold value.

8. The method of claim 1, wherein the threshold amount is determined, in part, as a threshold variance indicating one relevancy score is an outlier derived from a collection of relevancy scores for the number of phenotypes.

9. The method of claim 1, wherein the step of identifying the number of phenotypes, further comprises, applying a Hidden Markov Model (HMM) to obtain predictions of molecular entities associated with ones of the variant sites.

10. The method of claim 1, further comprising:
ranking entries of the phenotypes by difference between ones of the phenotypes and an associated difference between the subject and the background population.

11. The method of claim 10, further comprising extracting allele identification as a higher ranked phenotype from the ranked entries of the phenotypes.

12. The method of claim 1, wherein the step of comparing the degree of match between each pair of genomic data for relevant phenotypes, further comprises, deriving a score utilizing TF-IDF.

13. The method of claim 1, wherein the step of comparing the degree of match between each pair of genomic data for relevant phenotypes, further comprises:
comparing a distance between each pair of genomic data; and
deriving eigenvalues and engenvectors from the comparison of the distance between each pair of genomic data.

14. The method of claim 13, wherein:
at least one of the individual organisms and the background population comprise a cohort; and
the distance is determined by the formula:
Score=Local_score+μ·Global_score;
where, μ is a correction for cluster size for a cluster determined by the formula:
$$μ = \frac{{e}^{( γ \cdot \frac{cohort \_ size - cluster \_ size}{cohort \_ size} )} - 1}{{e}^{γ} - 1} ;$$
and
wherein score is a distance between genomic data of the at least one individual and genomic data of the background population;
wherein local_score comprises an average Euclidian distance from at least one individual from the cluster comprising the at least one individual;
wherein global_score comprises an average Euclidian distance for ones of the number of individual organisms within the cluster to all other of the at least one individual;
wherein cohort_size comprises the number of the at least one individual in the cohort; and
wherein cluster size comprises the number of the at least one individual in the cohort in the cluster.

15. The method of claim 1, wherein:
an individual and the background population comprise a cohort; and
score is determined by the formula:
score=(x*y*z)^{1/3};
wherein:
$$x = \frac{{r}^{1 + \frac{rank}{n}}}{r} ;$$
wherein:
$$y = \frac{eigenscore}{s} ;$$
wherein:
$$z = \frac{eigenscore - m 1}{m 2} ;$$
wherein: r=e¹⁵⁰;
wherein m1 is the minimum score of the individual in the cohort;
wherein m2 is the maximum score of the individual in the cohort;
wherein n is the number of individuals in the cohort;
wherein s is the sum of all eigenscores in the cohort; and
wherein rank is the rank of the individual within the background population.

16. The method of claim 1, further comprising administering a treatment to the subject based upon the report.
